(19) United States
(12) Plant Patent Application Publication
(10) Pub. No.: US 2012/0090067 P1
(43) Pub. Date: Apr. 12, 2012

(54) EXOTIC ORCHID 'Blue Birds'

(50) ;

(76) Inventor: Robert Glen Barfield, Kea'au, HI (US)

(21) Appl. No.: 12/924,793

(22) Filed: Oct. 6, 2010

(51) Int. Cl. A01H 5/00

(52) U.S. Cl. PLT/311

(57) ABSTRACT

A new variety of orchid plant of the Zygopetalinae Intergeneric group named EXOTIC ORCHID ‘Blue Birds’, distinguished in part by a multi hued violet fading to blues, and growing very fast to maturity without cultural difficulties from in-vitro culture to flowering.

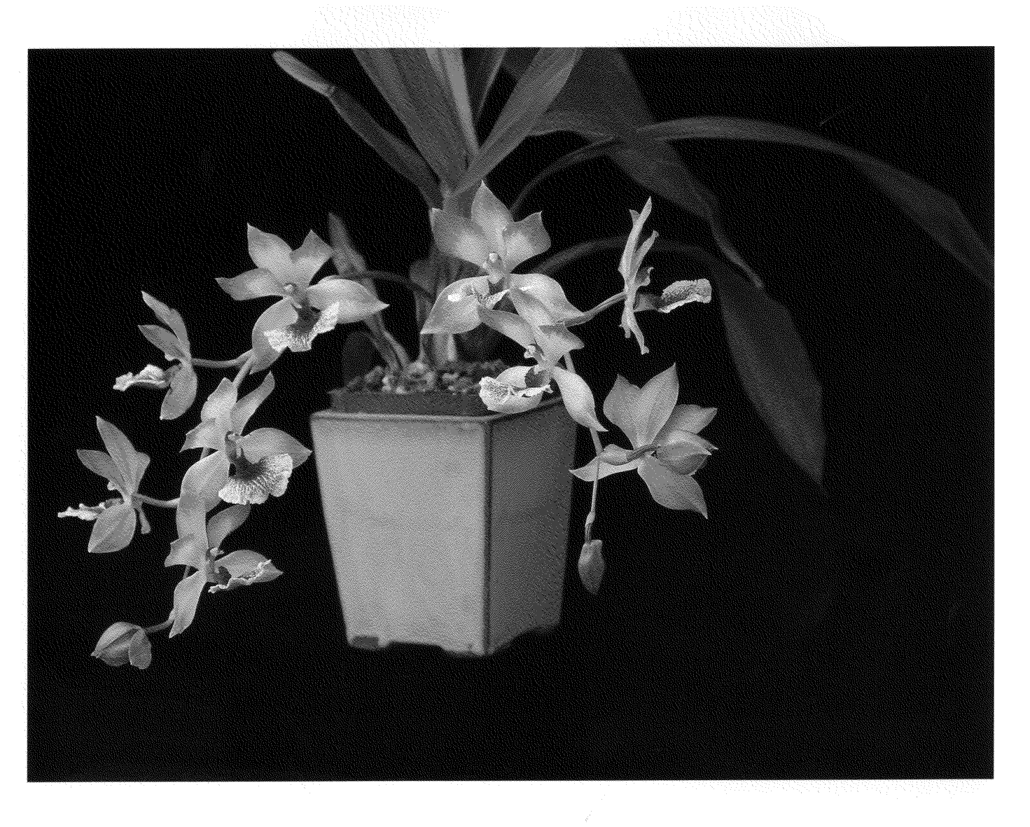

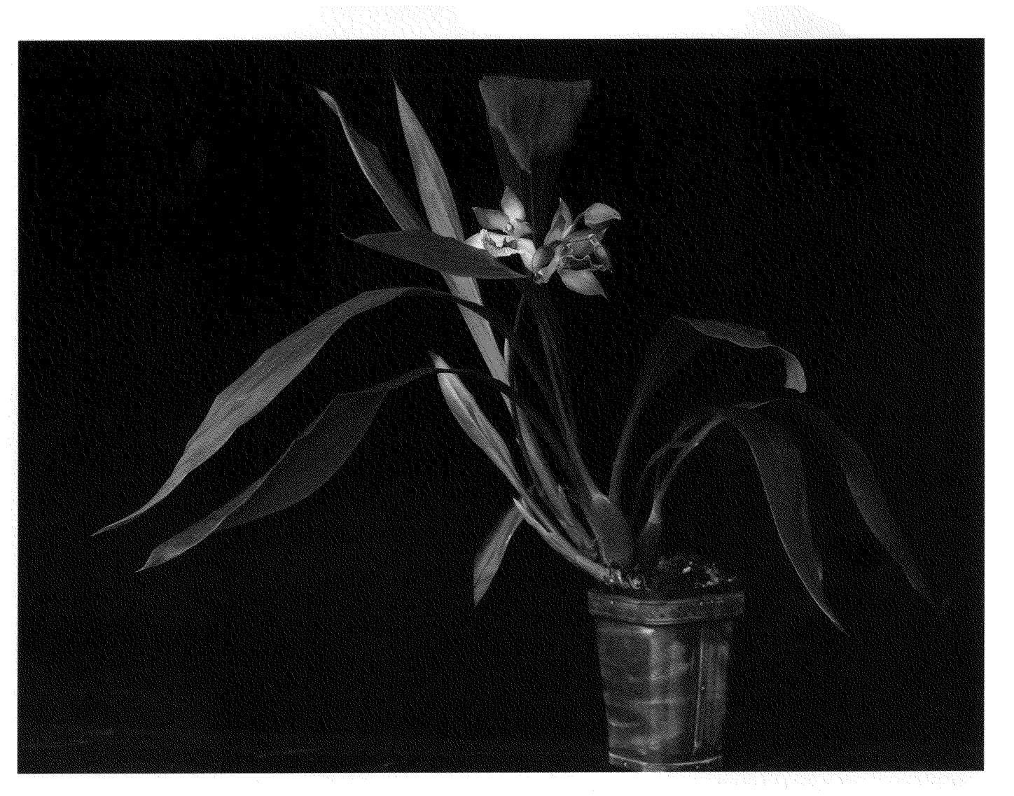

### CROSS-REFERENCE TO RELATED APPLICATIONS

Not Applicable

### STATEMENT REGARDING FEDERALLY SPONSORED RESEARCH OR DEVELOPMENT

Not applicable

### LATIN NAME OF THE GENUS AND GREX OF THE PLANT CLAIMED

The Latin name of the genus and grex of the plant claimed is Zygonisia Cynosure ‘Blue Birds’.

### VARIETY DENOMINATION

The present invention comprises a new and distinct cultivar of Zygopetalinae intergeneric orchid, and hereinafter referred to by the trade name EXOTIC ORCHID ‘Blue Birds’.

### BACKGROUND OF THE INVENTION

The sub-tribe Zygopetalinae is comprised of the genera Aganisia, Bollea, Chondrorhyncha, Cochleanthea, Huntleya, Kefersteinia, Koellensleinia, Pabstia, Pescatorea, Promenaea, Stenia and Zygopetalum all of which amount to approximately 143 species. These genera come from the tropical and subtropical South and Central Americas. Zygopetalinae are epiphytic, lithophytic and terrestrial. All species are Sympodial in growth and may vary greatly in their morphology and size.

Zygopetalinae breeding is typically done by sexual methods. Asexual propagation of Zygopetalinae is often done in aseptic tissue culture from apical and axillary shoots.

The new cultivar was discovered by the inventor within the progeny of a cross made by an unknown breeder. The seed was sewn Jun. 3, 2001 by the inventor, Glen Barfield dba Okika. EXOTIC ORCHID ‘Blue Birds’ was flowered, re-flowered several times, evaluated and determined to be worthy of production and protection. EXOTIC ORCHID ‘Blue Birds’ was submitted by the inventor to a commercial laboratory in Bangkok, Thailand on Apr. 6, 2007, for propagation through aseptic tissue culture technique. A quantity was produced for evaluation and has demonstrated that the unique combination of characteristics as herein disclosed for the new cultivar are firmly fixed and are retained through successive generations of asexual reproduction.

### BRIEF SUMMARY OF THE INVENTION

The following traits have been repeatedly observed and are determined to be basic characteristics of the new cultivar which in combination distinguish this Zygonisia orchid as a new and distinct cultivar.

EXOTIC ORCHID ‘Blue Birds’ has open flower segments with tepals having a light ocean foam green background early in flower life and overlaid with light blue centrally and a darker violet blue distally. The labellum is centrally dark violet with 7 keels on the callous and becomes blotchy light blue distally. The labellum is perpendicular to the plane of the tepals. The flower's natural spread is 6.3 centimeters horizontally and 6 centimeters vertically on first blooming plants.

Inflorescence is produced as a basal raceme, approximately 33 centimeters in length with up to 7 flowers per inflorescence. Each pseudobulb may carry 1 to 4 inflorescences. The inflorescences appear as upright and then slowly drop to just past horizontal when a full complement of flowers (6 to 7) is fully open, if not artificially supported.

The plant blooms during the growth of the first mature pseudobulb. There are typically 4 leaves on a mature pseudobulb which range in width from 3.5 centimeters to 6.9 centimeters and from 32 centimeters to 41 centimeters in length.

Plants of the new cultivar have not been observed under all possible environmental conditions. The phenotype may vary with variations in the environment such as temperature, light intensity, day length and nutrition. Subsequent bloomings will increase the size and number of flowers and inflorescences without, however, any change in genotype.

Plants of the new cultivar differ primarily from plants of the parent cultivars in flower size, color, inflorescence size, overall plant size and morphology, rate of growth, and speed to maturation. The closest commercial comparison to the new cultivar can be made to Zygonisia seedling orchids that are genetically heterogeneous and typically lack uniformity in growth, vigor, plant habit, and flower quality. Since this reference point has inconsistent characteristics, a direct comparison for EXOTIC ORCHID ‘Blue Birds’ is not currently available. The new cultivar is a single genotype asexually propagated via tissue culture; thus, its combined horticultural characteristics listed above are uniform and predictable.

### BRIEF DESCRIPTION OF THE DRAWINGS

All color references are measured against the Pantone® Color System. Colors and numerical measurements are approximate as plant growth and development depend on environmental conditions and cultural practices such as light level, temperature, water, nutrition, among others; without, however, any variance in genotype.

Plants used for this description are approximately 21 months in vivo and grown in 72 cell plug trays for 9 months and then in 3.5 inch, square plastic pots to flowering. The plants were grown in a poly-carbonate covered greenhouse near Hakalau, Hi., where day temperatures range from 73 to 88 degrees and night temperatures range from 62 to 75 degrees Fahrenheit. Light levels are between 1500 and 2500 foot candles. This information reflects the annual variations for the area.
Botanical classification: Zygonisia Cynosure ‘Blue Birds’
Parentage: Seedling selected from a cross of the following plants:
   Seed parent.—Zygopetalum Skippy Ku.
   Pollen parent.—Aganisia cyanea.
Propagation:
   Type.—asexual propagation by aseptic tissue culture axillary shoot initiation.
   Time to initiate and elongate shoots in-vivo.—about 280 days.
   Time to produce fully rooted young plants.—about 360 days.
Plant description: Under appropriate growing conditions, plants of the new cultivar attain a mature size of about 50 centimeters in height (top of leaf plane) and about 45 centimeters in width.
Root description: White with a thick velum covering. The root is approximately 4 millimeters in diameter and the growing tip is green.
Plant description:
   Arrangement.—sympodial growth habit.
   Quantity of leaves per grow.—4.
   Leaf length.—32 to 41 centimeters.
   Leaf width.—3.5 to 6.9 centimeters.
   Leaf shape.—lanceolate.
   Pseudobulb shape.—ovate and laterally compressed.
   Pseudobulb dimensions.—approximately 2.3 centimeters wide and 8.2 centimeters tall.
   Foliage color.—The foliage color of both the top and the bottom of the leaf as well as the pseudobulb is Pantone® 575C (green). The color is consistent with no contrasting venation color noted.
   Inflorescence description.—Inflorescence is produced as a basal raceme, approximately 33 centimeters in length with up to 7 flowers per inflorescence and each pseudobulb may have 1 to 4 inflorescences. The inflorescences appear as upright and then slowly drop to past horizontal when a full complement of flowers (5 to 7) is fully open.
   Flower arrangement description.—the flowers are arranged with alternates opposing.
   Flower description.—EXOTIC ORCHID ‘Blue Birds’ has open flower segments with petals and sepals having a light ocean foam green background and overlaid with light blue centrally and a darker violet blue distally. The labellum is centrally dark violet with seven keels on the callous and becomes blotchy light blue distally. The labellum is perpendicular to the plane of the tepals. The flower's natural spread is 6.3 centimeters horizontally and 6 centimeters vertically.
   Flower dimensions.—lateral sepal is 1.7 centimeters wide and 3.5 centimeters long; dorsal sepals are 1.9 centimeters wide and 3.5 centimeters long; petals are 1.9 centimeters wide and 3.5 centimeters long; the labellum is 3.2 centimeters wide and 3.3 centimeters long.
   Flower coloration.—The coloration of the sepals and petals are uniform with the central portion being Pantone® 263U (lavender) fading into the distal portion, which is 2705U (medium violet). The labellum is centrally Pantone® 2745U (violet) breaking up into distal blotches of Pantone® 2705U (violet blue) and Pantone® 2726U (darker violet blue).
   Flower quantity.—between 6 and 15 flowers on the first flowering.
   Flower longevity.—5 to 6 weeks.
   Flowering season.—EXOTIC ORCHID ‘Blue Birds’ does not seem to have a flowering season. When a new growth matures, it flowers. Flowering has occurred at about 7 month intervals once the plant is mature.
   Fragrance.—EXOTIC ORCHID ‘Blue Birds’ has been recognized as having a light floral fragrance.
   Reproductive organs.—The stamen, style and stigma are fused into a single short structure called the column, possessing one terminal anther with pollen grains united in a pollinia, which are covered by an anther cap. The stigma is located under the column behind the pollinia. The ovary is inferior, with three carpels being present.
   Column.—The column is erect with wings on either side of the stigmatic cavity, 0.8 centimeters wide and 2.1 centimeters long. The underside color is white with linear blue lines.
   Pollinia.—2 yellow oval masses of pollen are present, approximately 1.5 millimeters in diameter.
   Stigma.—the stigmatic cavity is concave, rounded trapezoid, 3 millimeters by 4 millimeters and filled with a sticky viscous material.
   Ovary.—the ovary is approximately 3 centimeters long and 3 millimeters in diameter.
   Seed.—seed production, while attempted, has not been successful.
   Disease resistance.—resistance to known pathogens of Zygopetalinae has not been observed on plants grown under commercial production conditions.
   General observations.—Plants of EXOTIC ORCHID ‘Blue Birds’ produce a pleasing arrangement of flowers held on a semi-erect raceme. The flowers are long lasting and the plant grows quickly and flowers early.

What is claimed is:

1. A new and distinct variety of orchid plant named EXOTIC ORCHID ‘Blue Birds’, substantially as illustrated and described herein.
